(19) United States
(12) Patent Application Publication
(10) Pub. No.: US 2010/0112031 A1
(43) Pub. Date: May 6, 2010

(54) Compositions And Methods For Regulating Extracellular Matrix Production In Adipose Derived Cells

(75) Inventor: Adam J. KATZ, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/580,417

(22) Filed: Oct. 16, 2009

Related U.S. Application Data
(60) Provisional application No. 61/106,173, filed on Oct. 17, 2008.

(51) Int. Cl. A61F 2/00; C12N 5/02; A61K 35/12

(52) U.S. Cl. 424/423; 435/395; 424/93.7

(57) ABSTRACT

The present application provides compositions and methods for regulating ECM production in ASCs and for isolating and using the various ECM molecules. The present application further provides methods for inducing various growth factors, cytokines and stem cell markers.

### CROSS-REFERENCE TO RELATED APPLICATIONS

This application claims the benefit of U.S. Provisional Application Nos. 61/106,173, filed Oct. 17, 2008, the disclosure of which is incorporated herein by reference in its entirety.

### STATEMENT REGARDING FEDERALLY SPONSORED RESEARCH

This invention was made in part with United States Government support under National Institutes of Health Grant Nos. R21 HL72141. The United States Government has certain rights in the invention.

### BACKGROUND

Human Adipose-derived cells can be formulated into 3-dimensional multicellular aggregates (MAs) and maintained for prolonged periods in suspension culture. These MAs produce self-generated extracellular matrix including collagen and other ECM components, without the addition of exogenous matrix factors and without the need for animal derived products. The ECM can be generated using a patients own cells (i.e. autologous), or can be generated using other's cells (i.e., allogeneic). The resulting ECM aggregates can be further processed to devitalize the cellular components, to yield an acellular 3-D matrix or scaffold—producing what would be considered an acellular device by the FDA, and avoiding transplantation/transmission of cells and/or other potential organisms. The cells can be devitalized in any number of ways known in the art, such as by temperature (ex. heat shock, freeze-thaw), osmotic shock (ex. exposure to hypotonic or hypertonic solutions), ultraviolet light exposure, gamma irradiation, mechanical, chemical (ex. fixatives such as formaldehyde) or other similar methods, or combination of methods. In addition, the ECM components, such as collagen can be further extracted and/or purified, and/or further processed/stabilized (such as by cross-linking) prior to use.

### BACKGROUND

As disclosed herein the extracellular matrix produced form the adipose-derived 3-dimensional multicellular aggregates can be modified and/or isolated and used for various therapeutic purposes.

### SUMMARY OF THE INVENTION

The present invention provides compositions and methods useful for regulating production of extracellular matrix (ECM) molecules in adipose-derived stromal cells. The present invention further provides methods for isolating and using the ECM molecules.

ECM molecules produced according to the methods of the invention include, but are not limited to, collagen type I, collagen type III, collagen type VI, fibronectin, tenascin, decorin, biglycan, and MMPs. In one aspect, the ECM molecules are autologous. In another aspect, they are allogeneic. In one aspect, the invention provides complex mixtures of ECM molecules.

In one embodiment, the invention provides methods for removing cells from the ECM or for devitalizing the cellular components.

The compositions and methods of the invention useful for diagnostic and therapeutic purposes.

In one embodiment, adipose-derived stromal cells can be stimulated to produce ECM molecules in vitro in the absence of animal-derived products. In another embodiment, adipose-derived stromal cells can be stimulated to produce ECM molecules in vitro in the absence of serum.

In one embodiment, the ECM components can be generated in a three-dimensional complex or scaffold type structure. In one aspect, the three-dimensional complex comprises multicellular aggregates (MA). In one aspect, ECM and growth factors and cytokines are induced in adipose-derived stromal cells when induced to form MAs. The modular adipogenic construct includes cellular components with adipogenic potential (including adipose stem cells) and has a self-generated extracellular matrix. Furthermore, the modular adipogenic construct is serum-free, free of exogenous materials, xenogenic-free, and free of other synthetic components. This basic modular construct is prepared by harvesting adipose tissue from a mammalian subject, isolating adipose tissue-derived stromal cells from the harvested adipose tissue, and culturing the isolated cells in 3-dimensional multicellular aggregates in a controlled, reproducible fashion. Culturing of isolated cells means the in vitro culturing of the isolated adipose tissue-derived stromal cells using appropriate means and methods. In one embodiment the adipose tissue is isolated from a human and in one embodiment the adipose tissue is harvested from the same individual that will receive a subsequently formed 3-dimensional multicellular aggregate implant derived from the harvested adipose tissue.

The preparation of multicellular aggregate can be prepared as described herein and in previous patent applications: US Provisional Application Nos. 61/221,577, filed Jun. 30, 2009; 61/118,055, filed Nov. 26, 2008; 61/107,398, filed Oct. 22, 2008; 61/106,758, filed Oct. 20, 2008; U.S. patent application Ser. No. 12/444,412, filed Apr. 6, 2009 and International application no. PCT/US2009/033220, filed Feb. 5, 2009 (published on Aug. 13, 2009 as WO 2009/100219), the disclosures of which are incorporated herein by reference in their entirety. In accordance with one embodiment, harvesting adipose tissue means the surgical removal of adipose tissue from other tissues naturally associated tissues resulting in a substantial enrichment of adipose tissue. In one embodiment harvesting adipose tissue is conducted either by excision, or more commonly, by liposuction. Isolation of adipose tissue-derived stromal cells from adipose tissues includes enriching for stromal cells relative to the harvested adipose tissue. Isolation of stromal cells can be conducted using mechanical and/or chemical processes by which the adipose tissue-derived stromal cells are separated (isolated) from the harvested adipose tissue. Typically the isolated stromal cells are cultured prior to formation of the 3-dimensional modular adipogenic constructs disclosed herein and such culturing steps include the in vitro culturing of the isolated adipose tissue-derived stromal cells using appropriate means and methods.

In accordance with one embodiment a method of regulating ECM production in adipose-derived stromal cells is provided. This method can be used to produce multiple ECM components, including collagens which are core component of tissue repair (especially skin and bone), which can be isolated from the underlying cells using standard techniques known to those skilled in the art. The method for preparing the adipose-derived stromal cell ECMs comprises the steps of

a. harvesting adipose tissue from a mammalian subject;

b. isolating adipose tissue-derived stromal cells from the harvested adipose tissue; and

c. culturing the isolated cells in 3-dimensional multicellular aggregates in a media selected to produce a desired extracellular matrix content. In one embodiment the isolated cells in 3-dimensional multicellular aggregates are cultured in the absence of non-human animal derived components. In one embodiment the isolated cells in 3-dimensional multicellular aggregates are cultured in serum free media. The content of the produced extracellular matrix (including the total concentration of collagen and other ECM components as well as their relative percent concentrations) can be further manipulated by selecting the composition of the media the isolated cells in 3-dimensional multicellular aggregates are cultured. For example, the isolated cells in 3-dimensional multicellular aggregates can be culture in the presence or absence of serum, in the presence of various vitamins, growth factors or other know bioactive factors to alter the composition of the resulting extracellular matrix secreted by the isolated cells in 3-dimensional multicellular aggregates. After preparation of the extracellular matrix, the isolated cells in 3-dimensional multicellular aggregates can optionally be removed using standard techniques to yield an acellular ECM product.

The 3-dimensional multicellular aggregate produced ECM can be prepared using autologous or off-the-shelf allogeneic paradigms and used to assist in the repair of damaged or diseased tissues in vivo. In accordance with one embodiment a method for inducing or enhancing the repair of damaged or diseased tissues is provided wherein 3-dimensional multicellular aggregate produced ECM is implanted into a patient. In one embodiment the 3-dimensional multicellular aggregate produced ECM is an acellular ECM product. Various therapeutic applications of the ECM components of the invention are described herein or would be understood by those of ordinary skill in the art.

Various aspects and embodiments of the invention are described in further detail below.

### BRIEF DESCRIPTION OF THE DRAWINGS

FIG. 1A-C: represent a series of bar graphs depicting the respective concentration of FN1 Isoform 1 of fibronectin precursor (FIG. 1A), TNC Isoform 1 of Tenascin precursor (FIG. 1B) and THBS1 Thrombospondin-1 precursor (FIG. 1C) in adipose-derived cells relative to multicellular aggregates of adipose-derived cells.

FIG. 2: is a bar graph showing the collagen content of the macrocellular aggregates (MAs) of Adipose Stem/Stromal Cells with and without vitamin C-induced collagen synthesis and without P-ascorbate. H8-08L FT denotes a human adipose stromal cell sample obtained from liposuction.

FIG. 3A-3D: provides standard curve analysis of sonicated MAs sample solutions.

### DETAILED DESCRIPTION

### Abbreviations and Acronyms

ASC—adipose-derived stromal cell

ECM—extracellular matrix

MA—multicellular aggregate

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease or disorder, including, but not limited to, pain and inflammation.

“Adipose-derived stem cells”, also referred to as “adipose-derived stromal cells” herein, refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, more preferably, the adipose tissue is human. A convenient source of adipose tissue is from liposuction surgery, however, the source of adipose tissue or the method of isolation of adipose tissue is not critical to the invention.

The term “adult” as used herein, is meant to refer to any non-embryonic or non-juvenile subject. For example the term “adult adipose tissue stem cell,” refers to an adipose stem cell, other than that obtained from an embryo or juvenile subject.

A disease or disorder is “alleviated” if the severity of a symptom of the disease, condition, or disorder, or the frequency with which such a symptom is experienced by a subject, or both, are reduced.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

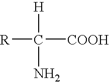

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “blastema”, as used herein, encompasses inter alia, the primordial cellular mass from which an organ, tissue or part is formed as well as a cluster of cells competent to initiate and/or facilitate the regeneration of a damaged or ablated structure.

The term “biocompatible,” as used herein, refers to a material that does not elicit a substantial detrimental response in the host.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “compound,” as used herein, refers to any type of substance or agent that is commonly considered a drug, or a candidate for use as a drug, combinations, and mixtures of the above, as well as polypeptides and antibodies of the invention.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The term “delivery vehicle” refers to any kind of device or material which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders,” and “feeder layers” are used interchangeably herein.

As used herein, a “functional” molecule is a molecule in a form in which it exhibits a property or activity by which it is characterized.

A “fragment” or “segment” is a portion of an amino acid sequence, comprising at least one amino acid, or a portion of a nucleic acid sequence comprising at least one nucleotide. The terms “fragment” and “segment” are used interchangeably herein.

“Graft” refers to any free (unattached) cell, tissue, or organ for transplantation.

“Allograft” or “allogeneic” refers to a transplanted cell, tissue, or organ derived from a different animal of the same species.

“Xenograft” or “xenogeneic” refers to a transplanted cell, tissue, or organ derived from an animal of a different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “ingredient” refers to any compound, whether of chemical or biological origin, that can be used in cell culture media to maintain or promote the proliferation, survival, or differentiation of cells. The terms “component,” “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, means to suppress or block an activity or function such that it is lower relative to a control value. The inhibition can be via direct or indirect mechanisms. In one aspect, the activity is suppressed or blocked by at least 10% compared to a control value, more preferably by at least 25%, and even more preferably by at least 50%.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

The term “injury” refers to any physical damage to the body caused by violence, accident, trauma, or fracture, etc.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

Used interchangeably herein are the terms: 1) “isolate” and “select”; and 2) “detect” and “identify”.

The term “isolated,” when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “low adherence, ultra low adherence, or non-adherence surface for cell attachment” refers to the ability of a surface to support attachment of cells. The term “non-adherence surface for cell attachment” means that the surface supports little if any cell attachment.

The term “modulate”, as used herein, refers to changing the level of an activity, function, or process. The term “modulate” encompasses both inhibiting and stimulating an activity, function, or process.

The terms “multicellular aggregate”, “multicellular sphere”, “blastema”, and “multicellular structure” are used interchangeably herein.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

The term “progeny” of a stem cell as used herein refers to a cell which is derived from a stem cell and may still have all of the differentiation abilities of the parental stem cell, i.e., multipotency, or one that may no longer be multipotent, but is now committed to being able to differentiate into only one cell type, i.e., a committed cell type. The term may also refer to a differentiated cell.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “solid support” when used in reference to a substrate forming a linkage with a compound, relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

The term “spawn”, as used herein, refers to the ability of the multicellular spheres of cells disclosed herein (SOMBs) to generate adherent cells (i.e., progeny) with the ability, inter alia, to grow to confluence.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or differentiation is stimulated by at least 10% compared to a control value, more preferably by at least 25%, and even more preferably by at least 50%. The term “stimulator” as used herein, refers to any compound or agent, the application of which results in the stimulation of a process or function of interest, including, but not limited to, ASC cell production, differentiation, and activity, as well as that of ASC progeny.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “substituent” as used in the phrase “other cells which are not substituents of the at least one self-organizing blastema” refers to substituent cells of the blastema. Therefore, a cell which is not a substituent of a self-organizing blastema can be a cell that is adjacent to the blastema and need not be a cell derived from a self-organizing blastema.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

### Embodiments

Methods useful for the practice of the invention which are not described herein are also known in the art. Useful methods include those described in WO 2007/030652 (PCT/US2006/034915), WO 2007/019107 (PCT/US2006/029686), WO 2007/089798 (PCT/US2007/002572), and WO 2008/060374 (PCT US2007/021432), the methods of which are hereby incorporated by reference.

### EXAMPLES

### Example 1

We have characterized the production of ECM components within human ASC MAs under a variety of growth conditions using histochemical, molecular and protein techniques. For instance, we have confirmed the presence of multiple types of collagen, including types I and III. Additional ECM components detected include fibronectin, tenascin, collagen VI, decorin, biglycan, and MMPs to name a few (see data included).

The ECM aggregates or ECM components produced as described have several potential commercial uses:

for research purposes (ex. as research reagents);

for therapeutic purposes including tissue augmentation/filling (such as the use of collagen to fill wrinkles in aged skin), or ECM to enhance/accelerate wound healing or tissue repair, etc.;

possibly for diagnostic purposes.

ECM components are known to be critical to normal biological process and tissue repair mechanisms. For example, many are known to potentiate or reduce the biological effect of important soluble growth factors (Marci et al., Clark et al.)

Some novel aspects of this invention:

generation of ECM from human adipose-derived cells

generation of ECM without the use/addition/need of animal derived products (such as FBS or bovine collagen, etc.)

multiple ECM components generated, including collagens which are core component of tissue repair (especially skin and bone)

ECM can be produced under a variety of conditions, including serum-free conditions

Cells that generate the ECM can subsequently be devitalized, to yield an acellular ECM product;

ECM can be prepared using autologous or off-the-shelf allogeneic paradigms

The type and amount of ECM generated can be varied depending on the culture media and growth conditions used.

### Example 1

Adipose-derived stem cells (ASCs) have the ability to form a spheroid body of macrocellular aggregates in a hanging drop suspension, which enables easy transfer/transplant of the cells in aggregates in contrast to the cells in monolayer. Thus, the possibility to induce collagen synthesis in ASCs in a three dimensional structure and a subsequent auto-transplant in a patient may provide a good alternative method for future skin reconstructive therapy. In this paper, the vitamin C-phosphate induced-collagen synthesis of ASCs was assessed by quantification of the collagen by the sirius red F3B dye binding assay and by the evaluation of the picro-sirius red staining of the cryosection. The results showed an increase of collagen production in VitC induced ASCs in 1% human serum media with approximately 29.4 μg of collagen per 200 k cells whereas about 13.4 μg of collagen per 200 k cells was measured in ASCs with no serum media and no VitC induction. Furthermore, the evaluation of the picro-sirius red staining under crossed-polarized bright field microscope showed increased production of Type I collagen fibers in VitC induced ASCs with serum media. Therefore, this study demonstrated that serum and VitC are two significant factors in order to induce increase in the collagen synthesis of the ASCs in a three dimensional structure.

Multicellular Aggregates

Our lab has developed techniques to culture human ASCs in suspension as 3-dimensional multicellular aggregates (MAs). Our previous studies have demonstrated the fabrication of a self-generated extracellular matrix (ECM) by ASCs formulated as MAs, such that a defined, manipulatable structure (‘organoid’) is generated. In contrast to cells grown as adherent monolayers, MAs enable the easy transfer/transplant of cells and ECM without disruption of cell-cell and cell-matrix interactions. The goal of this study was to confirm the presence of collagen in the extracellular matrix of ASC MAs and to quantify the relative amount of collagen production under varying culture conditions.

To reproducibly form cell aggregates, ASCs (500-50,000) were suspended in the appropriate medium to achieve desired concentrations. Small volumes (15-30 μl) of the concentrated cell suspensions were then pipetted onto culture plate covers in discrete droplets. The culture plate covers were then inverted, creating “hanging droplets”. After 24-72 hours in hanging drop culture, the MAs were then transferred to a range of media in either Ultra Low Attachment (ULA) wells/plates (Corning) for suspension culture, or into standard culture ware for adherent culture. In some experiments, ASC-MAs were labeled with Hoechst 33342 (Molecular Probes Cat#H1399) to reveal distribution of cell nuclei.

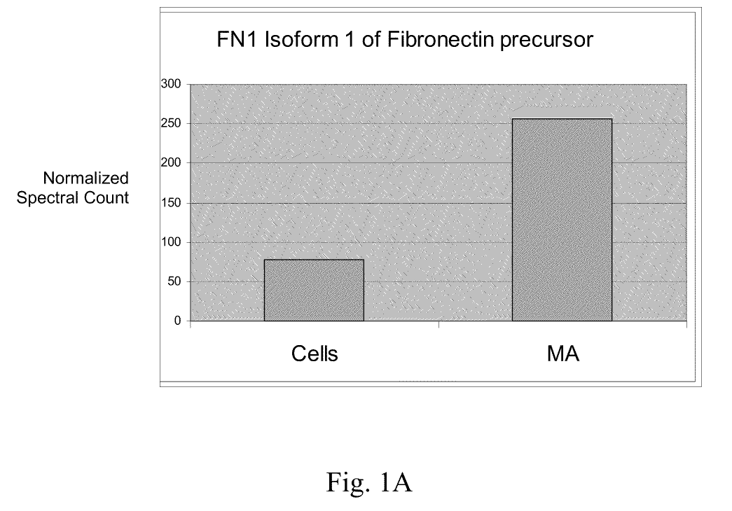

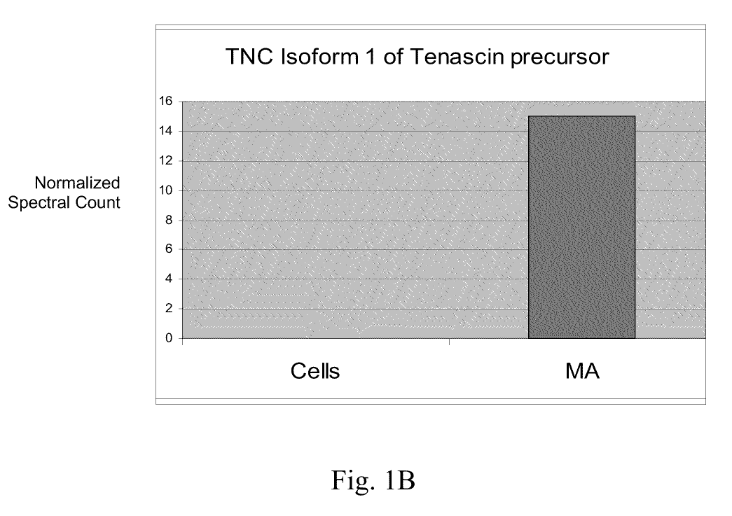

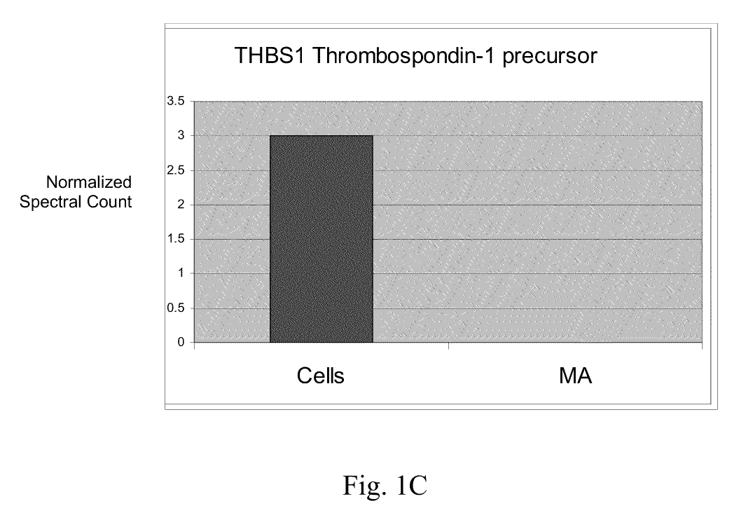

Results: Using a gravity-mediated technique, we demonstrate the successful and reproducible formation of 3-D ASC aggregates (MAs) using varied numbers of early passage ASCs (ranging from 500 to 50,000) isolated and cultured from individual donors (N>40). ASC MAs form, survive and grow in a variety of media types, including DMEM/F12 with 10% FBS (D-10), DMEM/F12 without serum or additives (D-0), serum-free ASC medium (AR8 and AR9), and low serum ASC medium (AR-1% HS and AR9-1% HS). FIG. 1a demonstrates the initial clustering and appearance of a typical ASC-MA soon after formation using a hanging drop technique. FIG. 1b demonstrates a photomicrograph of multiple well-defined, uniform sized MAs composed of fluorescently labeled (DiI) ASCs soon after their transfer to suspension culture.

Sirius Red F3B Dye Assay

Sirius Red F3B has been widely used in histological staining in order to identify collagen. The dye's ability to specifically bind to the collagen, however, can also be exploited to assess the amount of the collagen present in a given sample. The specificity of the dye is derived from the dye's ability to recognize and bind to the [gly-x-y] pattern of the triple helical structure of the collagen fiber. In addition, the elongated molecular structure of the dye interacts with the linear collagen fibers in a parallel fashion, and will not interact with other conformations of the collagen, i.e. denatured collagen [2]. Moreover, the anionic side chain groups of the dye and the basic amino acids in the collagen offer ionic interactions that enable stable dye-bindings throughout the assay.

Crossed Polarization Microscopy of Picrosirius Red Staining for Collagen Type I and III

The cross polarization microscopy utilizes the anisotropic property of the Sirius red dye bound-collagen fibers to identify the type I and III collagens. In contrast to the poor and variable staining of the thin collagen fibers of the van Gieson's stain, Puchtler et al. found that the Sirius Red stain with the yellow picric acid background can consistently stain the thin collagen fibers to give green color and the thicker type I collagen fibers to give red to orange color [3, 4].

Methods

Cell Culture

Cryopreserved human ASCs (obtained from liposuction) were thawed and plated using established protocols. Cells were cultured as adherent monolayers in LADP medium with 1% human serum until confluency. When the cell plates were confluent, the media were switched to LADP medium with no serum and incubated for two additional days. ASC MAs of 10⁵ cells were then fabricated in LADP medium with no serum and maintained in suspension culture in one of three different media (LADPM with 1% human serum (LADPM-1%), LADPM with no serum (LADMP-SF), and DMEM/F12 with antibiotics only (D0)). Each of these study arms was further divided into parallel cultures with or without 1 mM Ascorbic Acid-phosphate [1]. Media was changed on culture day 3, and MAs were harvested and analyzed on culture day 5 using two methods: Sirius Red F3B dye binding assay (detects all/most types of collagen) and picro-sirius red staining of cryosections (specific for Type I and 3 collagen fibers).

Collagen Sample Extraction

In preparation of the ASC MAs for Sirius Red F3B dye binding assay, the samples were first washed with PBS buffer three times. Three of ASC MAs of 10⁵ cells were then placed in 300 μl of extraction buffer: 0.5M Acetic Acid with 1:100 Protein Inhibitor Cocktail (Sigma Aldrich, St Louis, Mo., USA). The ASC MAs in the buffer solution was then sonicated three times, each for 5 seconds. If the extracellular matrix of the MAs were still visible, additional sonication was applied to disintegrate the structure. 200 μl of the sonicated sample solutions to yield approximately 200 k cells were used for the binding assay.

Sirius Red F3B Dye Binding Assay

Sterile bovine acid-soluble Type I Collagen of 1 mg/ml in 0.5M acetic acid solution (Biocolor Ltd, Belfast, Northern Ireland) was used to establish the standard curve. The known mass of the reference collagen (0, 19.5, 37.5, 75 μg) were mixed in 300 μl of the extraction buffer and sonicated accordingly. 200 μl of the standard collagen and the sample solutions were then mixed with 1 ml of Sirius Red Dye solution (1 mg/ml Direct Red 80, Sigma Aldrich, in 0.5 M acetic acid with 0.1% TWEEN 20) for 30 minutes. The samples were then centrifuged down at 13.2 k g for 10 minutes. Supernatants were removed without disrupting the pellet by decanting and tapping the tube on soft tissue paper, and the dye-bound sample pellets were resuspended in 1 ml of 0.5 M Sodium Hydroxide solution. After 5 minutes, 200 μl of the sample solutions were transferred to a 96 well plate and their absorbance was read at 540 nm

Cross Polarization Microscopy

The MAs were embedded and cryosectioned to 5 μm thickness. The cryosections were washed with deionized water and stained with the picrosirius red solution (0.1% Sirius red in saturated picric acid) for 1 hr [4]. The stained sample was then washed in two different 0.5 M acetic acid solutions, cleared in xylene, and mounted in synthetic resin. The pictures were captured with an Olympus BH-2 digital microscope camera with and without linear cross polarization.

### RESULTS AND DISCUSSION

In this study, the collagen content of the macrocellular aggregates (MAs) of Adipose Stem/Stromal Cells with and without vitamin C-induced collagen synthesis was investigated, and the result showed that serum as well as vitamin C, were two important factors in increasing the collagen content of the extracellular matrix (ECM) of the MAs. More specifically serum, or an unknown agent within serum, was important in increasing the total collagen (regardless of the type) measured by the Sirius Red F3B dye binding assay, and Vitamin C, in conjunction with the serum, was important in increasing the proportion of the Type I collagen in the increased total collagen content in the MAs.

One of the challenges of this study was to develop an effective extrication method to prepare the MAs for the dye-binding assay. Unless the dye can penetrate the ECM freely to bind to the collagen, the assay would be unfeasible without disrupting the ECM first. Preliminary tests have shown that the assay without sample extraction only yields the dye binding with the surface of the MAs and absorbance results that correlates the surface area, or the size of the MAs (result is not included in this paper). The conventional way of collagen extraction is to use pepsin digestion in 0.5 M acetic acid solution. The enzymatic approach to collagen extraction, however, did not disrupt ECM successfully even after 24 hr incubation of the sample with the pepsin solution while applying vigorous mix. Instead, a mechanical approach with probe sonication yielded positive results. Although a small amount of sample solution was vaporized, or expelled out of the tube, this loss was accounted with sampling 200 μl of 300 μl of the solutions. Furthermore, although the MAs in developmental period were maintained in a sterile environment, the MAs were then analyzed in non-sterile environment after harvesting the samples on day 5. Therefore, the empiric use of the proteinase inhibitor cocktail warranted significant sample yield without any sample loss from the opportunistic bacterial protease exposure. In addition, the possibility of mechanically, or thermally denaturing the collagen was accounted with the standard curve. The R value of the standard curve varied from 0.96 to 0.99.

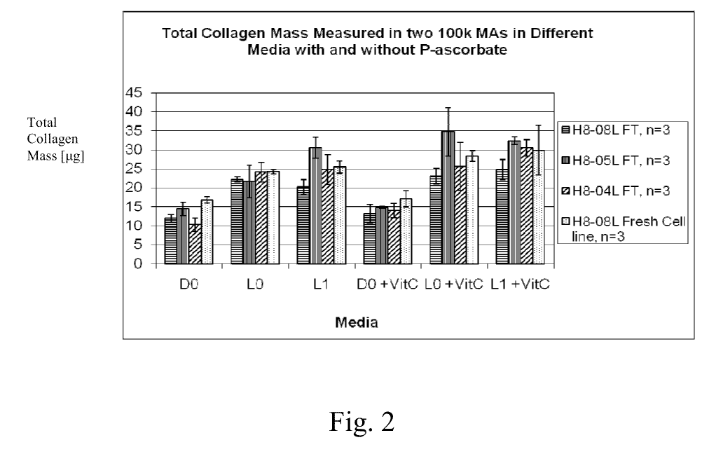

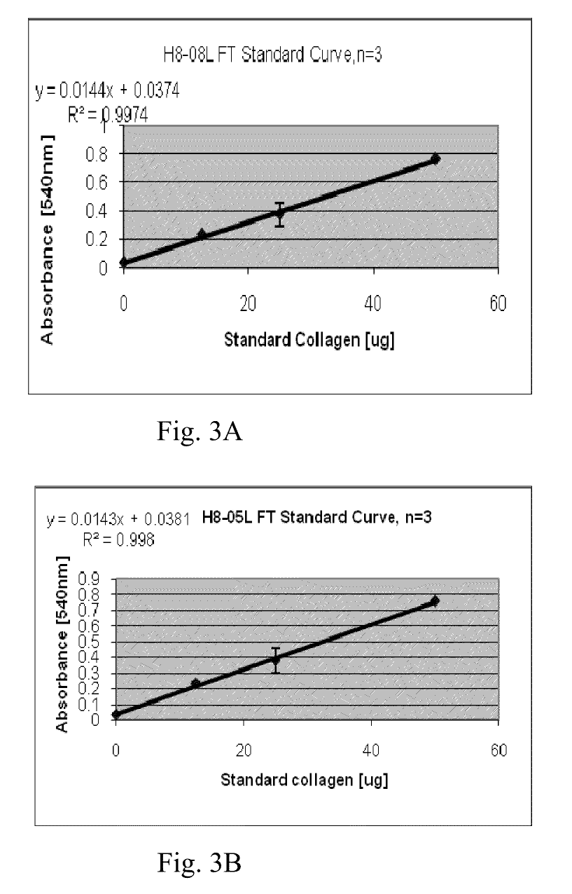

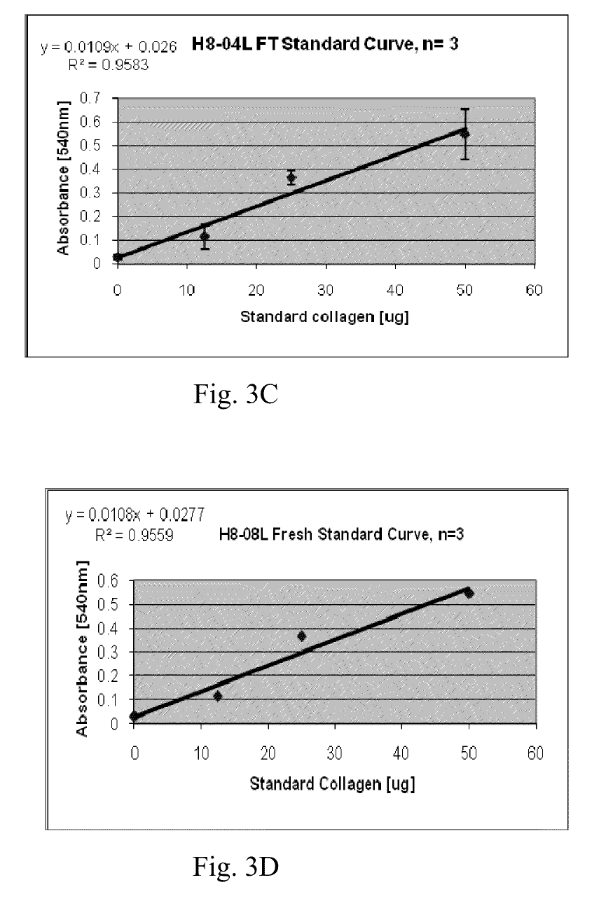

Sonication of the MAs yielded sample solutions suitable for the Sirius Red F3B dye-binding assay; however, such a process also resulted in possible degradation of the collagen in the solutions. This possible degradation of collagen fibers in the sample is reflected in the standard curve of the H8-04L FT and H8-08L Fresh (FIGS. 3A-3D). These two patient samples still had few MAs intact after initial sonication procedure; therefore, the second round of sonication procedure was applied. Thus, the R value of the standard curve decreased from 0.99 of samples (H8-08L FT, H8-05L FT) which only had one round of sonication procedure to completely disrupt the MAs to 0.96 of R value from undergoing two rounds of sonication procedure. This, however, does not seem to affect the result of the data significantly, as shown in FIG. 2 and Table 1. All four patient samples showed similar data within standard deviation.

The four patient data showed ASC MAs generated detectable collagen under all culture conditions, but more collagen was generated in LADP medium than DMEM medium. Under serum free conditions in DMEM medium, the addition of vitamin C did not significantly increase collagen levels. In the presence of serum, however, vitamin C increased collagen production by ˜20% (Table 2). It is interesting to note that LADP medium with no serum and LADP medium with 1% human serum showed similar results although LADP medium with serum showed higher collagen content. LADP medium is a highly defined medium in that every agent/molecular content is predetermined. It is speculated whether one of the LADP agents is responsible for the increased production of collagen synthesis, just as an agent in serum would encourage collagen synthesis.

In addition, the growth kinetic of the MAs cannot be ignored. It is known that medium with serum promotes more cell proliferation, which may correlate with increased production of ECM. Perhaps, the larger proportion of increased total collagen content in MAs incubated with LADP medium with serum and P-ascorbic acid can be attributed to increased cellular proliferation.

The significance of the vitamin C induction is not quite apparent until the content of the ECM of the MAs can be observed. The visualization of picro-sirius red stained MA sections under crossed-polarized bright field microscope showed the increased production of Type I collagen in conditions with vitamin C and serum. Significant amounts of type I collagen (red and orange) and type III collagen (green) can be visualized in MAs with vitamin C induction [3]. Picrosirius stained collagen fibers are more pronounced as it glows from the dark background in crossed polarized microscopy.

Conclusions:

This study demonstrates that ASCs produce self-generated collagen when formulated as defined multicellular aggregates in suspension. Even more, ASC MAs support collagen production in defined, serum-free conditions. Based on these findings, ASC MAs may prove useful for therapeutic applications that would benefit from collagen supplementation/replacement.

### Example 1

### Bibliography

1. Boyera N, Galey I, Bernard BA. (1998) Effect of vitamin C and its derivatives on collagen synthesis and cross-linking by normal human fibroblasts. Intern. Journ. Of Cosmetic Sci. 20, 151-158
2. Lee D. A., Assoku E, Doyle V (1998) A specific quantitative assay for collagen synthesis by cells seeded in collagen based biomaterials using Sirius red F3B precipitation J. of Materials Sci.: Materials in Medicine 9, 47-51
3. Otto J, Kammer D, Jansen P L, Anurov M, Titkova S, Ottinger A, Rosch R, Schumpelick V, Jansen M (2008) Different Tissue reaction of oesophagus and diaphragm after mesh hiatoplasty. Results of an animal study. BMC surgery 2008, 8:7
4. Whittaker P, Rich L (2005) Collagen and picrosirius red staining: a polarized light assessment of fibrillar hue and spatial distribution. Braz. J. Morphol. Sci 22(2), 97-104

### Example 1

### Appendix

### Example 1

FIG. 2: H8-08L FT denotes Human Adipose Stromal Cell sample obtained from liposuction and was frozen in single cell suspension with cryoprotectant. The cryopreserved cells were thawed and plated in LADP medium with 1% human serum.

TABLE 1

| | | H8-08L FT, | | H8-05L FT, | | H8-04L FT, | | H8-08L Fresh | |
|---|---|---|---|---|---|---|---|---|---|
| | | n = 3 | | n = 3 | | n = 3 | | Cell line, n = 1 | |
| | Media | Mass [μg] | Std | Mass [μg] | Std | Mass [μg] | Std | Mass [μg] | Std |
| Control | D0 | 11.99 | 1.04 | 14.47 | 1.76 | 10.25 | 1.7 | 16.82 | 0.84 |
| | L0 | 22.22 | 0.65 | 21.67 | 4.3 | 24.1 | 2.52 | 24.3 | 0.61 |
| | L1 | 20.25 | 2 | 30.55 | 2.78 | 24.83 | 3.93 | 25.43 | 1.67 |
| Vit C | D0 + VitC | 13.14 | 2.43 | 14.86 | 0.21 | 13.98 | 1.91 | 17.07 | 2.09 |
| | L0 + VitC | 23.12 | 2.1 | 34.77 | 6.4 | 25.63 | 6.36 | 28.33 | 1.32 |
| | L1 + VitC | 24.76 | 2.65 | 32.37 | 1.02 | 30.55 | 2.17 | 29.9 | 6.56 |

TABLE 2

| | Serum | Vitamin | Collagen | Standard |
|---|---|---|---|---|
| Medium | (1% HS) | C | (μg/200k MA) | Deviation |
| LADPM | + | + | 29.4 | 4.34 |
| LADPM | + | − | 25.27 | 4.47 |
| LADPM | − | + | 27.96 | 6.04 |
| LADPM | − | − | 23.07 | 2.47 |
| DMEM/F12 | − | + | 14.76 | 2.21 |
| DMEM/F12 | − | − | 13.38 | 2.86 |

### Example 2

Introduction: Emerging evidence supports the therapeutic potential of Adipose-Derived Stem Cells (ASCs) in the healing of cutaneous wounds. Using a murine model of delayed diabetic wound healing, our team has demonstrated enhanced in vivo potency of ASCs formulated as 3-dimensional multicellular aggregates (MAs), as compared to ASCs grown as adherent monolayers and delivered as single cell suspensions. The purpose of this study was to elucidate transcriptional and translational differences between the two cell formulation strategies that may provide mechanistic insights into the basis for our in vivo findings.

Background

It is estimated that 1% of the world's population will develop a chronic wound at some point in their lifetime. 12% of hospital admissions are related to the treatment of these wounds and these account for 21% of total inpatient hospital days.

Most chronic wounds such has diabetic ulcers have been found to have altered cell milieu which in turn leads to impaired cell migration, proliferation, adhesion, growth factor production and signaling.

Adipose derived stem cells have been shown to not only be induced in to multiple cell types but have also been shown to secrete multiple growth factors, and cytokines such as hepatocyte growth factor (HGF), vascular endothelial growth factor (VEGF), fibroblast growth factor (FGF), and interleukins. Many of these proteins have been shown to improve wound healing rates in both in vitro and in vivo models.

In recent in vivo experiments using rat wound healing models, we have compared the effects of ASCs delivered as multicellular aggregates versus ASCs delivered as cell suspension. Equal numbers of ASCs were delivered using the two dosing systems. MA delivery models showed improved wound healing rates when compared to cell suspension delivery models.

Materials and Methods

Human ASCs were isolated and plated using established techniques. Cells were expanded in adherent monolayer culture in growth factor enriched medium with 1% human serum (AR8-1%). After sufficient expansion, half of the cells were formed into 3-D MAs and maintained in suspension culture, and half were maintained in adherent monolayer culture. On day 6, cells or culture medium from each group were harvested and analyzed by one of three techniques depending on the specific experiment: 1) microarray analysis (3 donors); 2) ELISA analysis (1 donor); and 3) mass spectrometry (1 donor).

Microarray Analysis

Human ASCs were harvested from 3 different donors (Donor 1: 34 y/o F, BMI:25.1; Donor 2: 45 y/o F, BMI:28.2; Donor 3:31 y/o F, BMI:35.4)

The cells were expanded as adherent monolayers in AR 8/9-1% HS medium until sufficient numbers were obtained.

One half of the cells were lifted and placed back into monolayer culture at 2,000 cells/cm², while the remainder were used to prepare Multicellular Aggregates (MA)@25 k ASCs/MA.

On days 2-3 fresh medium was provided for all cultures. Between days 5-7, cells were harvested from both groups and RNA was isolated using a commercially available kit.

Monolayers and MAs from the 3 donors were analyzed in triplicate (N=9/group) using separate/individual Affymetrix human gene chips (HgU133 plus 2.0).

An agilent BioAnalyzer was used to analyze the quality of each total RNA sample. 260/280 spectrophotometer absorbance readings were measured for both total RNA and biotinylated cRNA to rule out protein contamination, presence of degraded RNA, truncated cRNA transcripts and or excess free nucleotides.

Background intensity was derived from the intensity values of the lowest 2% of cells on the chip and was subtracted from all cells before gene expression levels were calculated.

Mass Spectrometry Analysis

Analysis using liquid chromatography mass spectrometry with a Finnigan LTQ-FT system and Protana nanospray ion source was carried out on the monolayer and 3D ASC populations.

Samples analyzed were ASCs grown as monolayer and 3D MAs in A1 medium. 10 μL volumes of prepared protein-gel extract were injected and peptides eluted from the column by an acetonitrile/acetic acid gradient at a flow rate of 0.25 μL/min over 2 hours. No exogenous ECM proteins were added to the samples and the proteins expressed by the cell and MA populations on days 3 and 6 were compared. The data was analyzed using the Sequest search algorithm against Human International Protein Index

Results

Microarray analysis revealed the statistically significant upregulation of at least 85 genes by a factor of 2 fold or greater (p<0.05). Upregulated genes included IGF-1 (57×), BGN (21×), IGFBP (31×), PDGF (16×) VCAM1 (14×) MMP-1 (14×), TNC(12×); HGF (11×) among others. On categorizing these genes, we found gene expression patterns demonstrating a profile reflective of tissue repair, ECM remodeling, wound healing, keratinocyte migration and angiogenesis (5) We also observed an upregulation in skin/hair follicle stem cell markers which included Sox9 (8×), TCF₃ (2×), NFATc (1.5×) (4). Upregulated genes that are specifically pertinent to diabetic wound healing include, VEGF, TIMP-1 TIMP-2, MMP-1, PDGF, TGFB1, IGF-1,1,2,3 ELISA and mass spectrometry analysis confirmed the translational upregulation of most of these genes.

In order to explain these improved wound healing rates in MA architecture, microarray analysis was used to compare the gene expression profiles of the ASC-MAs to the ASC monolayers. We decided to use the following categories in order to classify and compare the gene expression:

Synergistic Signalling

One of the reasons we see enhanced wound healing with our multicellular aggregates is due to the presence of growth factors as well as extracellular matrix molecules in their niche environments. Growth factors and ECM matrix proteins have been shown to interact with each other to either amplify or retard healing of wounds. These observations have lead to the proposition that when used in synergistic combinations with ECM molecules, the amount of growth factors needed to display a given effect on wound healing would be much lower than if they are used in non-synergistic combinations (Clark, 2008).

Synergistic Combinations

Decorin—IGF-1→Repository, ↑ local concentration of IGF-1→↑ chemotactic activity in endothelial cell lines, ↑ keratinocyte and fibroblast proliferation and re-epithelialization

Decorin—TGFB1→↑ angiogenesis, stimulation of PDGFA 4→↑ chemotaxis (neutrophils, monocytes, fibroblasts), ↑ proliferation (fibroblasts), induction of myofibroblast phenotype.

↑

Biglycan—TGF beta→Repository, ↑ local concentration

Fibronectin—VEGF, TGF beta→Repository and longer activation period→↑ angiogenesis, chemotaxis, proliferation.

Fibronectin—HGF→Repository and longer activation period→↑ endothelial migration.

Tenascin—EGF receptors→improved mitogenic and migratory activity

Thrombospondin—VEGF→internalization of VEGF→delayed granulation tissue formation and diminished angiogenesis→delayed wound healing.

| Gene Symbol | Fold Change | Description |
|---|---|---|
| IGF1 | 57.2816 | Insulin-like growth factor 1 |
| BGN | 21.11213 | Biglycan |
| TNC | 12.99604 | Tenascin C |
| HGF | 11.31371 | Hepatocyte growth factor |
| VEGF | 1.866066 | Vascular endothelial growth factor |
| TGFB1 | 1.283426 | Transforming growth factor, beta 1 |
| DCN | 1.239708 | Decorin |
| FN1 | 1.079228 | Fibronectin 1 |

Keratinocyte Migration

Keratinocyte motility depends on two major extracellular cues. ECM, which promotes haptotaxis and essential for initiation of motility and GFs that are essential for chemotaxis and augmenting the action of ECM molecules. Studies have shown upregulation of certain promotility genes in response to growth factor stimulation. When compared to our data, the following promotility genes are upregulated by at least a factor of two.

| Gene Symbol | Fold Change | Description |
|---|---|---|
| ID1 | 5.133704 | Inhibitor of DNA binding 1 |
| ARL4C | 4.257481 | ADP-ribosylation factor-like 4C |
| LIF | 4.198867 | Leukemia inhibitory factor |
| ZFP36L2 | 3.340352 | Zinc finger protein 36, C3H type-like 2 |
| JUN | 3.09513 | v-jun sarcoma virus 17 oncogene homolog |
| SLC20A1 | 2.479415 | Solute carrier family 20 member 1 |
| IL11 | 2.462289 | Interleukin 11 |
| IER2 | 2.297397 | Immediate early response 3 |
| NALP1 | 2.114036 | NACHT, leucine rich repeat and PYD |
| | | (pyrin domain) containing 1 |

Epithelial-Mesenchymal Interaction

In addition to activation of promotility genes in keratinocytes, wound healing process involves epithelial-mesenchymal interaction between keratinocytes and fibroblasts. Keratinocyte proliferation and migration of fibroblasts to the wound site begins at the end of the inflammatory phase. Keratinocytes at the wound site mediate gene expression changes in fibroblasts and thereby affect leukocyte attraction and adhesion, fibroblast and keratinocyte proliferation, angiogenesis and ECM remodeling. Corresponding genes that are upregulated in our microarray data are as follows

| Gene Symbol | Fold Change | Description |
|---|---|---|
| IGF1 | 57.2816 | insulin-like growth factor 1 |
| MMP1 | 13.92881 | matrix metallopeptidase 1 |
| SPON1 | 8.224911 | spondin 1, extracellular matrix protein |
| PTGS2 | 7.061624 | prostaglandin-endoperoxide synthase 2 |
| NR4A2 | 5.656854 | nuclear receptor subfamily 4, group A, |
| | | member 2 |
| PTGES | 5.133704 | prostaglandin E synthase |
| IER3 | 5.063026 | immediate early response 3 |
| ENO2 | 4.287094 | enolase 2 |
| JUNB | 4.228072 | jun B proto-oncogene |
| KYNU | 3.506423 | kynureninase |
| NR4A3 | 3.317278 | nuclear receptor subfamily 4, group A, |
| | | member 3 |
| PDE4D | 3.138336 | phosphodiesterase 4D |
| FMOD | 3.010493 | fibromodulin |
| PDXK | 2.732081 | pyridoxal kinase |
| CA12 | 2.713209 | carbonic anhydrase XII |
| TGFBR1 | 2.584706 | transforming growth factor, beta receptor I |
| TNFAIP6 | 2.479415 | tumor necrosis factor, alpha-induced |
| | | protein 6 |
| IL11 | 2.462289 | interleukin 11 |
| CA12 | 2.281527 | carbonic anhydrase XII |
| GK | 2.084932 | glycerol kinase |
| NDRG1 | 2.056228 | N-myc downstream regulated gene 1 |

Stem Cell Niche Contributions

Stem cell niches in adult mammalian skin are found to be present in the intrafollicular dermis, in sebaceous glands and in the bulge region of the outer root sheath of the hair follicle. Studies have shown that this bulge region contributes multipotent cells that are preferentially tapped in cases of cutaneous damage. Not much is known about the manner in which the stem cells reach the bulge in the embryonic stage, but the gene Sox9 is known to play a critical role in stem cell specification. Progeny of Sox9 producing cells contribute to all skin layers. Other transcription factors that are upregulated in bulge stem cells include Lhx2, Tcf3, and Nfatc1. The ones that are expressed in our data are:

| Gene Symbol | Fold Change | Description |
|---|---|---|
| SOX9 | 7.94474 | SRY (sex determining region Y)-box 9 |
| TCF3 | 2.07053 | Transcription factor 3 |
| NFATC1 | 1.453973 | Nuclear factor of activated T-cells |

Conclusions: ASCs prepared as 3-D MAs statistically up-regulate the expression of many important factors involved in wound healing and tissue repair, including soluble growth factors and extracellular matrix proteins. Given the emerging evidence for the synergistic bio-activity of ECM-growth factor complexes, these findings may help explain the enhanced potency of 3-D MAs that we have observed related in vivo studies.

### Example 2

### Bibliography

1. S. Rahman, et al., Novel hepatocyte growth factor (HGF) binding domains on fibronectin and vitronectin coordinate a distinct and amplified Met-integrin induced signalling pathway in endothelial cells, BMC Cell Biol. 6 (1) (2005) 8.
2. C. Cabello-Verrugio, E. Brandan, A novel modulatory mechanism of TGF-beta signaling through decorin and LRP-1, J. Biol. Chem. (2007).
3. R. Blakytny, et al., Lack of insulin-like growth factor 1 (IGF1) in the basal keratinocyte layer of diabetic skin and diabetic foot ulcers, J. Pathol. 190 (5) (2000) 589-594.
4. C. S. Swindle, et al., Epidermal growth factor (EGF)-like repeats of human tenascin-C as ligands for EGF receptor, J. Cell Biol. 154 (2) (2001) 459-468.
5. M. Streit et. al., Thrombospondin-1 suppresses wound healing and granulation tissue formation in the skin of transgenic mice. EMBO 19 (2000) 3272-82.
6. R. A. F. Clark, Synergistic Signalling from Extracellular Matrix-Growth Factor Complexes, J. Inv. Dermatology. 128 (2008) 1354-1355.
7. Marci, Lauren et. al. “Growth factor binding to the pericellular matrix and its importance in tissue engineering”, Advanced Drug Delivery Reviews 59 (2007) 1366-1381.
8. Nowinski, D. et. al. “Analysis of Gene Expression in Fibroblasts in Response to Keratinocyte-Derived Factors In Vitro: Potential Implications for the Wound Healing Process”, J Invest Dermatol 122 (2004) 216-221.
9. Chen et. al. “Profiling Motility Signal-Specific Genes in Primary Human Keratinocytes”, J Invest Dermatol 128 (2008) 1981-1990.

### Example 3

### Methods

Cryopreserved human ASCs were thawed and plated using established protocols. Cells were cultured as adherent monolayers in LADP medium with 1% human serum until confluency. ASC MAs of 10⁵ cells were then fabricated and maintained in suspension culture in one of three different media (LADPM with 1% human serum (LADPM-1%), LADPM with no serum (LADMP-SF), and DMEM/F12 with antibiotics only (D0)). Each of these study arms was further divided into parallel cultures with or without Ascorbic Acid-phosphate. Medium was changed on culture day 3, and MAs were harvested and analyzed on culture day 5 using two methods: Sirius Red F3B dye binding assay (detects all/most types of collagen) and picro-sirius red staining of cryosections.

Results: ASC MAs generated detectable collagen under all culture conditions, but more collagen was generated in LADP medium than DMEM medium. Under serum free conditions, the addition of vitamin C did not significantly increase collagen levels. In the presence of serum, however, vitamin C increased collagen production by ˜20% (Table). Visualization of picro-sirius red stained MA sections under crossed-polarized bright field microscope showed the presence of Type I and III collagen fibers.

| | Serum | Vitamin | Collagen |
|---|---|---|---|
| Medium | (1% HS) | C | (μg/100k MA) |
| LADPM | + | + | 20 |
| LADPM | + | − | 16.5 |
| LADPM | − | + | 15.5 |
| LADPM | − | − | 17.5 |
| DMEM/F12 | − | + | 14 |
| DMEM/F12 | − | − | 12.5 |

Conclusions:

This study demonstrates that ASCs produce self-generated collagen when formulated as defined multicellular aggregates in suspension. Even more, ASC MAs support collagen production in defined, serum-free conditions. Based on these findings, ASC MAs may prove useful for therapeutic applications that would benefit from collagen supplementation/replacement.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

1. A method of making a 3-dimensional modular cellular construct, the modular cellular construct comprising adipose-derived stromal cells, said method comprising the steps of:
a. harvesting adipose tissue from a mammalian subject;
b. isolating adipose tissue-derived stromal cells from the harvested adipose tissue;
c. culturing the isolated cells in 3-dimensional multicellular aggregates in a controlled, reproducible fashion such that the 3-dimensional multicellular aggregate self-generates an extracelluar matrix that is serum-free, free of exogenous materials, zenogeneic-free, and free of synthetic components; and
d. inducing the adipose tissue-derived stromal cells in culture to differentiate to produce a collagen, decorin, fibronectin, biglycan, elastin, tenascin C, osteonectin, or osteopontin.

2. The method of claim 1 wherein the adipose-derived stromal cells comprise cells selected from the group consisting of adipose stem cells, stromal cells, and progenitor cells.

3. A method of using 3-dimensional modular cellular constructs for therapeutic purposes, including tissue repair, tissue regeneration, tissue replacement, or tissue augmentation, said method having the steps of:
a. generating 3-dimensional modular cellular constructs that have a self-generated extracelluar matrix that is serum-free, free of exogenous materials, zenogeneic-free, and free of synthetic components, and that has been induced in culture to differentiate to produce a collagen, decorin, fibronectin, biglycan, elastin, tenascin C, oteonectin, or osteopontin; and
b. implanting said modular constructs in the tissues of a patient.

4. The method of claim 3 wherein the modular cellular construct is combined with growth factors, antibiotics, or cytokines.

5. The method of claim 3 wherein the cellular construct is combined with a gel, scaffold, or exogenous factor.

6. A method of regulating ECM production in adipose-derived stromal cells, said method comprising the steps of
a. harvesting adipose tissue from a mammalian subject;
b. isolating adipose tissue-derived stromal cells from the harvested adipose tissue;
c. culturing the isolated cells in 3-dimensional multicellular aggregates in a media selected to produce a desired extracellular matrix content.

7. The method of claim 6 wherein the isolated cells in 3-dimensional multicellular aggregates are cultured in the absence of non-human animal derived components.

8. The method of claim 7 wherein the isolated cells in 3-dimensional multicellular aggregates are cultured in serum free media.

9. The method of claim 6 wherein the isolated cells in 3-dimensional multicellular aggregates are cultured in the presence of an exogenous source of vitamin C.
